(19) United States
(12) Patent Application Publication
(10) Pub. No.: US 2021/0174824 A1
(43) Pub. Date: Jun. 10, 2021

(54) Neural Network Audio Scene Classifier for Hearing Implants

(71) Applicant: MED-EL Elektromedizinische Geraete GmbH, Innsbruck (AT)

(72) Inventors: Rainer Martin, Bochum (DE); Semih Agcaer, Bochum (DE); Florian Frühauf, Rinn (AT); Ernst Aschbacher, Innsbruck (AT); Erhard Rank, Innsbruck (AT)

(21) Appl. No.: 17/263,068

(22) PCT Filed: Jul. 24, 2019
(86) PCT No.: PCT/US2019/043160

§ 371 Date: Jan. 25, 2021

Related U.S. Application Data
(60) Provisional application No. 62/703,490, filed on Jul. 26, 2018.

(51) Int. Cl. G10L 25/51; G10L 25/18; G10L 25/30; H04R 25/00

(52) U.S. Cl. CPC G10L 25/51; H04R 25/507; G10L 25/30; G10L 25/18

(57) ABSTRACT

An audio scene classifier classifies an audio input signal from an audio scene and includes a pre-processing neural network configured for pre-processing the audio input signal based on initial classification parameters to produce an initial signal classification, and a scene classifier neural network configured for processing the initial scene classification based on scene classification parameters to produce an audio scene classification output. The initial classification parameters reflect neural network training based on a first set of initial audio training data, and the scene classification parameters reflect neural network training on a second set of classification audio training data separate and different from the first set of initial audio training data. A hearing implant signal processor configured for processing the audio input signal and the audio scene classification output to generate the stimulation signals to the hearing implant for perception by the patient as sound.

This application claims priority from U.S. Provisional Patent Application 62/703,490, filed Jul. 26, 2018, which is incorporated herein by reference in its entirety.

### TECHNICAL FIELD

The present invention relates to hearing implant systems such as cochlear implants, and specifically to the signal processing used therein associated with audio scene classification.

### BACKGROUND ART

A normal ear transmits sounds as shown in FIG. 1 through the outer ear 101 to the tympanic membrane 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, hearing prostheses have been developed. For example, when the impairment is related to operation of the middle ear 103, a conventional hearing aid may be used to provide mechanical stimulation to the auditory system in the form of amplified sound. Or when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system, including an external microphone that provides an audio signal input to an external signal processor 111 where various signal processing schemes can be implemented. The processed signal is then converted into a digital data format, such as a sequence of data frames, for transmission into the implant 108. Besides receiving the processed audio information, the implant 108 also performs additional signal processing such as error correction, pulse formation, etc., and produces a stimulation pattern (based on the extracted audio information) that is sent through an electrode lead 109 to an implanted electrode array 110.

Typically, the electrode array 110 includes multiple electrode contacts 112 on its surface that provide selective stimulation of the cochlea 104. Depending on context, the electrode contacts 112 are also referred to as electrode channels. In cochlear implants today, a relatively small number of electrode channels are each associated with relatively broad frequency bands, with each electrode contact 112 addressing a group of neurons with an electric stimulation pulse having a charge that is derived from the instantaneous amplitude of the signal envelope within that frequency band.

It is well-known in the field that electric stimulation at different locations within the cochlea produce different frequency percepts. The underlying mechanism in normal acoustic hearing is referred to as the tonotopic principle. In cochlear implant users, the tonotopic organization of the cochlea has been extensively investigated; for example, see Vermeire et al., Neural tonotopy in cochlear implants: An evaluation in unilateral cochlear implant patients with unilateral deafness and tinnitus, Hear Res, 245(1-2), 2008 Sep. 12 p. 98-106; and Schatzer et al., Electric-acoustic pitch comparisons in single-sided-deaf cochlear implant users: Frequency-place functions and rate pitch, Hear Res, 309, 2014 March, p. 26-35 (both of which are incorporated herein by reference in their entireties).

In some stimulation signal coding strategies, stimulation pulses are applied at a constant rate across all electrode channels, whereas in other coding strategies, stimulation pulses are applied at a channel-specific rate. Various specific signal processing schemes can be implemented to produce the electrical stimulation signals. Signal processing approaches that are well-known in the field of cochlear implants include continuous interleaved sampling (CIS), channel specific sampling sequences (CSSS) (as described in U.S. Pat. No. 6,348,070, incorporated herein by reference), spectral peak (SPEAK), and compressed analog (CA) processing.

In the CIS strategy, the signal processor only uses the band pass signal envelopes for further processing, i.e., they contain the entire stimulation information. For each electrode channel, the signal envelope is represented as a sequence of biphasic pulses at a constant repetition rate. A characteristic feature of CIS is that the stimulation rate is equal for all electrode channels and there is no relation to the center frequencies of the individual channels. It is intended that the pulse repetition rate is not a temporal cue for the patient (i.e., it should be sufficiently high so that the patient does not perceive tones with a frequency equal to the pulse repetition rate). The pulse repetition rate is usually chosen at greater than twice the bandwidth of the envelope signals (based on the Nyquist theorem).

In a CIS system, the stimulation pulses are applied in a strictly non-overlapping sequence. Thus, as a typical CIS-feature, only one electrode channel is active at a time and the overall stimulation rate is comparatively high. For example, assuming an overall stimulation rate of 18 kpps and a 12 channel filter bank, the stimulation rate per channel is 1.5 kpps. Such a stimulation rate per channel usually is sufficient for adequate temporal representation of the envelope signal. The maximum overall stimulation rate is limited by the minimum phase duration per pulse. The phase duration cannot be arbitrarily short because, the shorter the pulses, the higher the current amplitudes have to be to elicit action potentials in neurons, and current amplitudes are limited for various practical reasons. For an overall stimulation rate of 18 kpps, the phase duration is 27 μs, which is near the lower limit.

The Fine Structure Processing (FSP) strategy by Med-El uses CIS in higher frequency channels, and uses fine structure information present in the band pass signals in the lower frequency, more apical electrode channels. In the FSP electrode channels, the zero crossings of the band pass filtered time signals are tracked, and at each negative to positive zero crossing, a Channel Specific Sampling Sequence (CSSS) is started. Typically CSSS sequences are applied on up to 3 of the most apical electrode channels, covering the frequency range up to 200 or 330 Hz. The FSP arrangement is described further in Hochmair I, Nopp P, Jolly C, Schmidt M, Schaer H, Garnham C, Anderson I, MED-EL Cochlear Implants: State of the Art and a Glimpse into the Future, Trends in Amplification, vol. 10, 201-219, 2006, which is incorporated herein by reference. The FS4 coding strategy differs from FSP in that up to 4 apical channels can have their fine structure information used. In FS4-p, stimulation pulse sequences can be delivered in parallel on any 2 of the 4 FSP electrode channels. With the FSP and FS4 coding strategies, the fine structure information is the instantaneous frequency information of a given electrode channel, which may provide users with an improved hearing sensation, better speech understanding and enhanced perceptual audio quality. See, e.g., U.S. Pat. No. 7,561,709; Lorens et al. “Fine structure processing improves speech perception as well as objective and subjective benefits in pediatric MED-EL COMBI 40+ users.” International journal of pediatric otorhinolaryngology 74.12 (2010): 1372-1378; and Vermeire et al., “Better speech recognition in noise with the fine structure processing coding strategy.” ORL 72.6 (2010): 305-311; all of which are incorporated herein by reference in their entireties.

Many cochlear implant coding strategies use what is referred to as an n-of-m approach where only some number n electrode channels with the greatest amplitude are stimulated in a given sampling time frame. If, for a given time frame, the amplitude of a specific electrode channel remains higher than the amplitudes of other channels, then that channel will be selected for the whole time frame. Subsequently, the number of electrode channels that are available for coding information is reduced by one, which results in a clustering of stimulation pulses. Thus, fewer electrode channels are available for coding important temporal and spectral properties of the sound signal such as speech onset.

In addition to the specific processing and coding approaches discussed above, different specific pulse stimulation modes are possible to deliver the stimulation pulses with specific electrodes—i.e. mono-polar, bi-polar, tri-polar, multi-polar, and phased-array stimulation. And there also are different stimulation pulse shapes—i.e. biphasic, symmetric triphasic, asymmetric triphasic pulses, or asymmetric pulse shapes. These various pulse stimulation modes and pulse shapes each provide different benefits; for example, higher tonotopic selectivity, smaller electrical thresholds, higher electric dynamic range, less unwanted side-effects such as facial nerve stimulation, etc.

Fine structure coding strategies such as FSP and FS4 use the zero-crossings of the band-pass signals to start a channel-specific sampling sequence (CSSS) pulse sequences for delivery to the corresponding electrode contact. Zero-crossings reflect the dominant instantaneous frequency quite robustly in the absence of other spectral components. But in the presence of higher harmonics and noise, problems can arise. See, e.g., WO 2010/085477 and Gerhard, David, Pitch extraction and fundamental frequency: History and current techniques, Regina: Department of Computer Science, University of Regina, 2003; both incorporated herein by reference in their entireties.

FIG. 2 shows an example of a spectrogram for a sample of clean speech including estimated instantaneous frequencies for Channels 1 and 3 as reflected by evaluating the signal zero-crossings, indicated by the vertical dashed lines. The horizontal black dashed lines show the channel frequency boundaries—Channels 1, 2, 3 and 4 range between 100, 198, 325, 491 and 710 Hz, respectively. It can be seen in FIG. 2 that during periods of a single dominant harmonic in a given frequency channel, the estimate of the instantaneous frequency is smooth and robust; for example, in Channel 1 from 1.6 to 1.9 seconds, or in Channel 3 from 3.4 to 3.5 seconds. When additional frequency harmonics are present in a given channel, or when the channel signal intensity is low, the instantaneous frequency estimation becomes inaccurate, and, in particular, the estimated instantaneous frequency may even leave the frequency range of the channel.

FIG. 3 shows various functional blocks in a signal processing arrangement for a typical hearing implant. The initial input sound signal is produced by one or more sensing microphones, which may be omnidirectional and/or directional. Preprocessor Filter Bank 301 pre-processes this input sound signal with a bank of multiple parallel band pass filters (e.g. Infinite Impulse Response (IIR) or Finite Impulse Response(FIR)), each of which is associated with a specific band of audio frequencies; for example, using a filter bank with 12 digital Butterworth band pass filters of 6th order, Infinite Impulse Response (IIR) type, so that the acoustic audio signal is filtered into some K band pass signals, U₁ to U_{K} where each signal corresponds to the band of frequencies for one of the band pass filters. Each output of sufficiently narrow CIS band pass filters for a voiced speech input signal may roughly be regarded as a sinusoid at the center frequency of the band pass filter which is modulated by the envelope signal. This is also due to the quality factor (Q≈3) of the filters. In case of a voiced speech segment, this envelope is approximately periodic, and the repetition rate is equal to the pitch frequency. Alternatively and without limitation, the Preprocessor Filter Bank 301 may be implemented based on use of a fast Fourier transform (FFT) or a short-time Fourier transform (STFT). Based on the tonotopic organization of the cochlea, each electrode contact in the scala tympani typically is associated with a specific band pass filter of the Preprocessor Filter Bank 301. The Preprocessor Filter Bank 301 also may perform other initial signal processing functions such as and without limitation automatic gain control (AGC) and/or noise reduction and/or wind noise reduction and/or beamforming and other well-known signal enhancement functions.

FIG. 4 shows an example of a short time period of an input speech signal from a sensing microphone, and FIG. 5 shows the microphone signal decomposed by band-pass filtering by a bank of filters. An example of pseudocode for an infinite impulse response (IIR) filter bank based on a direct form II transposed structure is given by Fontaine et al., Brian Hears: Online Auditory Processing Using Vectorization Over Channels, Frontiers in Neuroinformatics, 2011; incorporated herein by reference in its entirety.

The band pass signals U₁ to U_{K} (which can also be thought of as electrode channels) are output to an Envelope Detector 302 and Fine Structure Detector 303. The Envelope Detector 302 extracts characteristic envelope signals outputs Y₁, . . . , Y_{K} that represent the channel-specific band pass envelopes. The envelope extraction can be represented by Yₖ=LP (|Uₖ|), where |.| denotes the absolute value and LP(.) is a low-pass filter; for example, using 12 rectifiers and 12 digital Butterworth low pass filters of 2nd order, IIR-type. Alternatively, the Envelope Detector 302 may extract the Hilbert envelope, if the band pass signals U₁, . . . , U_{K} are generated by orthogonal filters.

The Fine Structure Detector 303 functions to obtain smooth and robust estimates of the instantaneous frequencies in the signal channels, processing selected temporal fine structure features of the band pass signals U₁, . . . , U_{K} to generate stimulation timing signals X₁, . . . , X_{K}. In the following discussion, the band pass signals U₁, . . . , Uₖ are assumed to be real valued signals, so in the specific case of an analytic orthogonal filter bank, the Fine Structure Detector 303 considers only the real valued part of Uₖ. The Fine Structure Detector 303 is formed of K independent, equally-structured parallel sub-modules.

The extracted band-pass signal envelopes Y₁, . . . , Y_{K} from the Envelope Detector 302, and the stimulation timing signals X₁, . . . , X_{K} from the Fine Structure Detector 303 are input signals to a Pulse Generator 304 that produces the electrode stimulation signals Z for the electrode contacts in the implanted electrode array 305. The Pulse Generator 304 applies a patient-specific mapping function—for example, using instantaneous nonlinear compression of the envelope signal (map law)—That is adapted to the needs of the individual cochlear implant user during fitting of the implant in order to achieve natural loudness growth. The Pulse Generator 304 may apply logarithmic function with a form-factor C as a loudness mapping function, which typically is identical across all the band pass analysis channels. In different systems, different specific loudness mapping functions other than a logarithmic function may be used, with just one identical function is applied to all channels or one individual function for each channel to produce the electrode stimulation signals. The electrode stimulation signals typically are a set of symmetrical biphasic current pulses.

### SUMMARY OF THE INVENTION

Embodiments of the present invention are directed to a signal processing system and method to generate stimulation signals for a hearing implant implanted in a patient. An audio scene classifier is configured for classifying an audio input signal from an audio scene and includes a pre-processing neural network configured for pre-processing the audio input signal based on initial classification parameters to produce an initial signal classification, and a scene classifier neural network configured for processing the initial scene classification based on scene classification parameters to produce an audio scene classification output. The initial classification parameters reflect neural network training based on a first set of initial audio training data, and the scene classification parameters reflect neural network training on a second set of classification audio training data separate and different from the first set of initial audio training data. A hearing implant signal processor configured for processing the audio input signal and the audio scene classification output to generate the stimulation signals to the hearing implant for perception by the patient as sound.

In further specific embodiments, the pre-processing neural network includes successive recurrent convolutional layers, which may be implemented as recursive filter banks. The pre-processing neural network may include an envelope processing block configured for calculating sub-band signal envelopes for the audio input signal. The pre-processing neural network also may include a pooling layer configured for signal decimation within the pre-processing neural network. The initial signal classification may be a multi-dimensional feature vector. The scene classifier neural network may be a fully connected neural network layer or a linear discriminant analysis (LDA) classifier.

### BRIEF DESCRIPTION OF THE DRAWINGS

FIG. 1 shows the anatomy of a typical human ear and components in a cochlear implant system.

FIG. 2 shows an example spectrogram of a speech sample.

FIG. 3 shows major signal processing blocks of a typical cochlear implant system.

FIG. 4 shows an example of a short time period of an input speech signal from a sensing microphone.

FIG. 5 shows the microphone signal decomposed by band-pass filtering by a bank of filters.

FIG. 6 shows major functional blocks in a signal processing system according to an embodiment of the present invention.

FIG. 7 shows processing steps in initially training a pre-processing neural network according to an embodiment of the present invention.

FIG. 8 shows processing steps in iteratively training a classifier neural network according to an embodiment of the present invention.

FIG. 9 shows functional details of a pre-processing neural network according to one specific embodiment of the present invention.

FIG. 10 shows an example of how filter bank filter bandwidths may be structured according to an embodiment of the present invention.

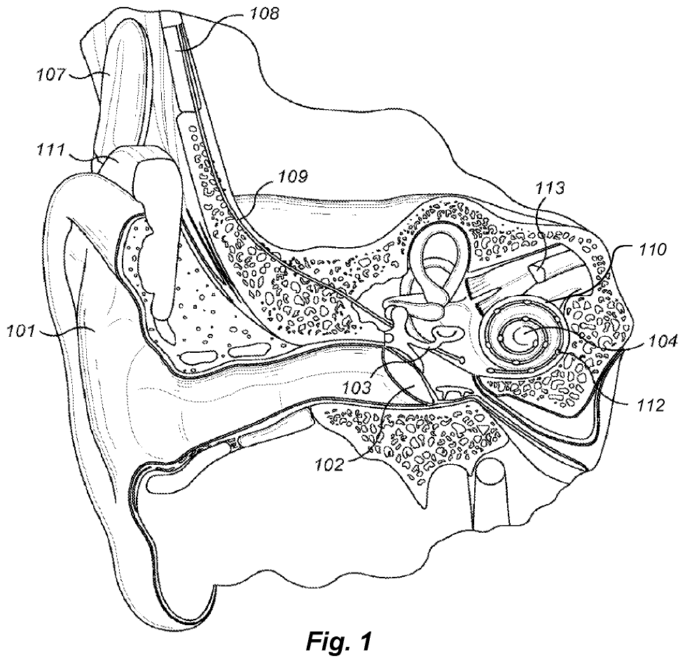

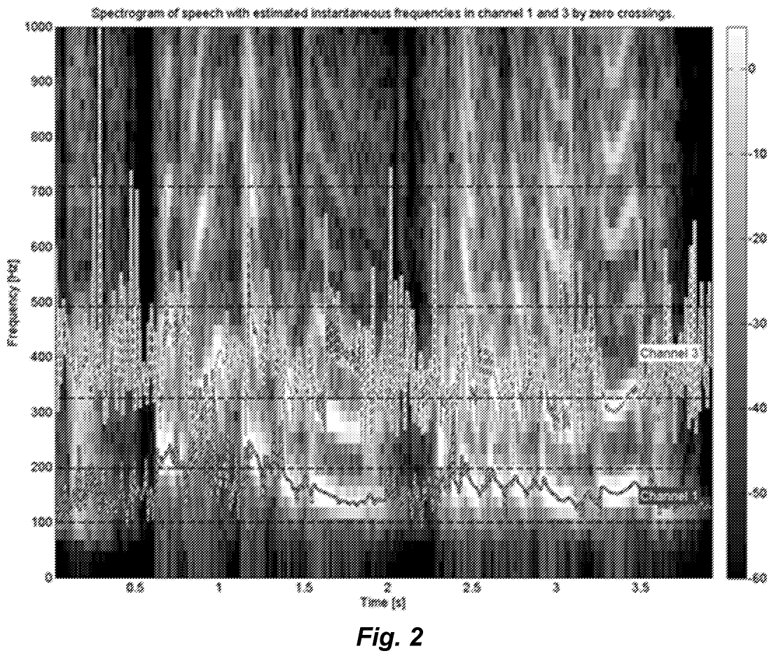

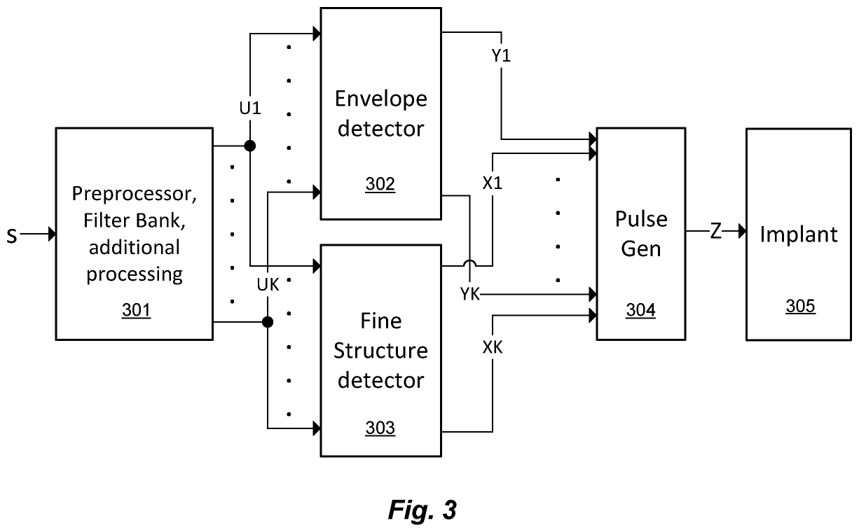

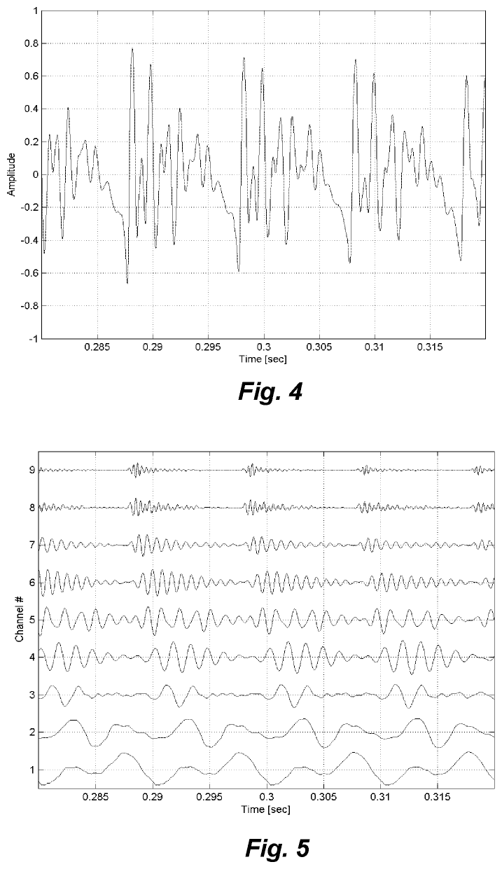

### DETAILED DESCRIPTION OF SPECIFIC EMBODIMENTS

Neural network training is a complicated and demanding process that requires a lot of training data for optimizing the parameters of the network. The effectiveness of the training further very much depends on the training data that is used. Many undesirable side effects may occur after the training, and it might even happen that the neural network does not even perform the intended task. This problem is particularly pronounced when trying to classify audio scenes for hearing implants where a nearly infinite number of variations exist for each classified scene and seamless transitions occur between distinct scenes.

Embodiments of the present invention are directed to an audio scene classifier for hearing implants that uses a multi-layer neural network optimized for iterative training of a low number of parameters that can be trained with reasonable effort and sized training sets. This is accomplished by separating the neural network into an initial pre-processing neural network whose output is then input to a classification neural network. This allows for separate training of the individual neural networks and thereby allows use of smaller training sets and faster training that is carried out in a two-step process as described below.

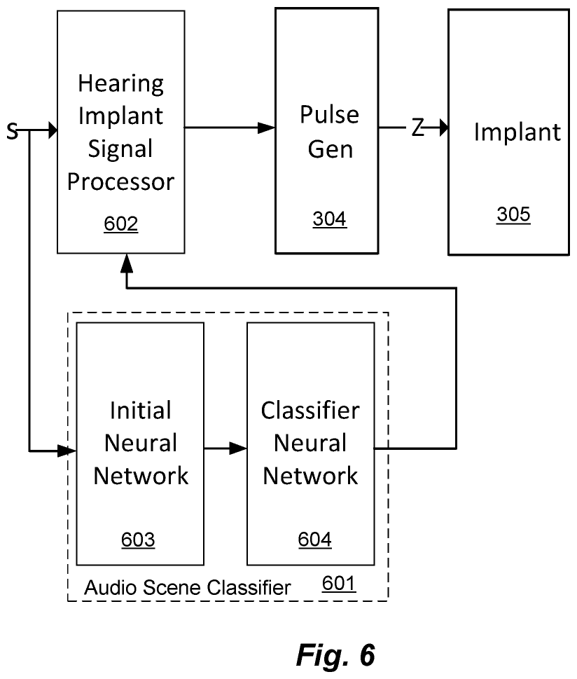

FIG. 6 shows major functional blocks in a signal processing system according to an embodiment of the present invention for generating stimulation signals for a hearing implant implanted in a patient. An audio scene classifier 601 is configured for classifying an audio input signal from an audio scene and includes a pre-processing neural network 603 that is configured for pre-processing the audio input signal based on initial classification parameters to produce an initial signal classification, and a scene classifier neural network 604 that is configured for processing the initial scene classification based on scene classification parameters to produce an audio scene classification output. The initial classification parameters reflect neural network training based on a first set of initial audio training data, and the scene classification parameters reflect neural network training on a second set of classification audio training data separate and different from the first set of initial audio training data. A hearing implant signal processor 602 is configured for processing the audio input signal and the output of the audio scene classifier 601 to generate the stimulation signals to a pulse generator 304 to provide to the hearing implant 305 for perception by the patient as sound.

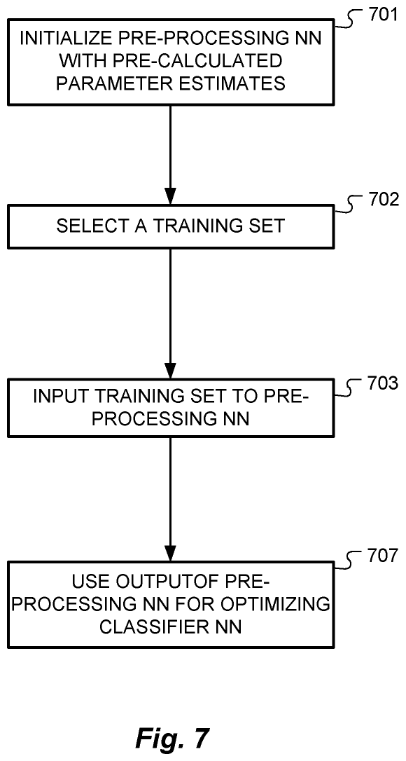

FIG. 7 shows processing steps in initially training the pre-processing neural network 603, which starts, step 701, by initializing the pre-processing neural network 603 with pre-calculated parameter that are within an expected range of parameters, for example, in the middle of a parameter range. A first training set of audio training data (Training Set 1) is selected, step 702, and input for training of the pre-processing neural network 603, step 703. The output from the pre-processing neural network 603 then, step 704, is used as the input to the classifier neural network 604 for optimizing it using various known optimization methods.

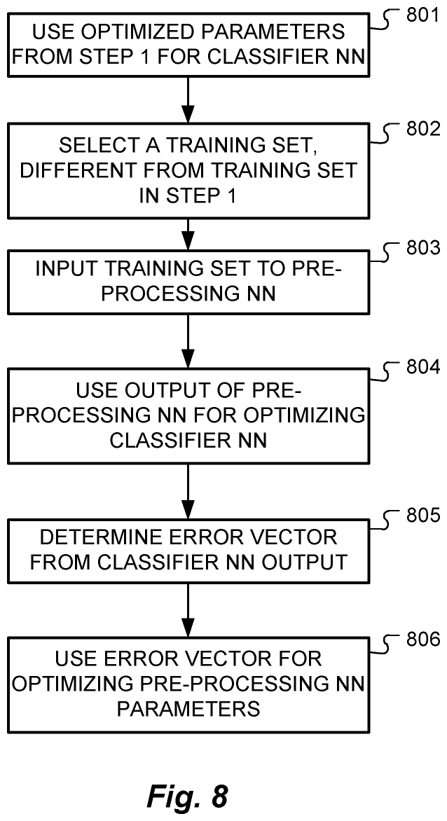

FIG. 8 then shows various subsequent processing steps in iteratively training a classifier neural network 604 starting with the optimized parameters from the initial training of the pre-processing neural network as discussed above with regards to FIG. 7, step 801. A second training set of audio training data (Training Set 2), which is different from the first training set, is selected, step 802, and input to the pre-processing neural network 603. The output from the pre-processing neural network 603 is further input and processed by the classification neural network 604, step 804. An error vector then is calculated, step 805, by comparing the output from the classification neural network 604 to the audio scene that the second training set data should belong to. The error vector then, step 806, is used to optimize the pre-processing neural network 603. The new parameterization of the pre-processing neural network 603, then leads to a two-step iterative training procedure that ends when selected stopping criteria are met.

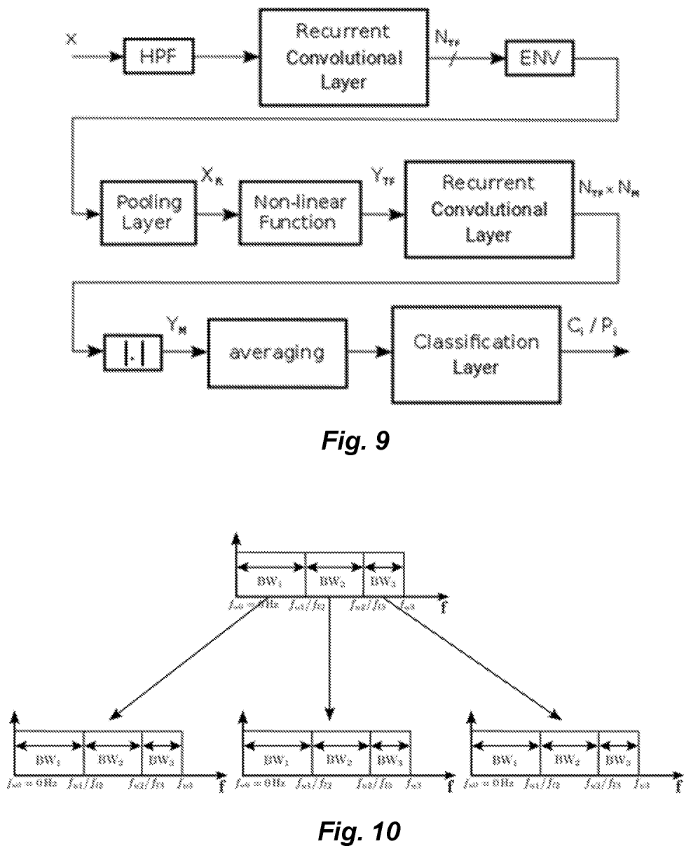

FIG. 9 shows functional details of a pre-processing neural network according to one specific embodiment of the present invention with several linear and non-linear processing blocks. In the specific example shown, there are two successive recurrent convolutional layers, pooling layers, non-linear functions and an averaging layer. The recurrent convolutional layers can be implemented as recursive filters banks. Without loss of generality, the input signal is assumed to be an audio signal x(k) with length N, which is first high-pass filtered (HPF-block) and then fed into N_{TF} parallel processing blocks that act as band pass filters. This leads to N_{TF} output sub band signals x_{T,i}(k) with different spectral contents. The band pass filtered sub band signals can be expressed by the equation:

$${x}_{T , i} ( k ) = {\sum }_{n = 0}^{{P}_{1}} {b}_{i , n} x ( k - n ) - {\sum }_{m = 1}^{{P}_{2}} {a}_{i , m} {x}_{T , i} ( k - m )$$

where b_{i,n} are the feed forward coefficients, and a_{i,n} the feedback coefficients of the i-th filter block. The filter order is P=max(P₁, P₂).

The sub band signals envelopes then are calculated by rectification and low pass filtering. Note that any other method for determining the envelopes can be used, too. The low pass filter may be, for example, a fifth-order recursive Chebyshev II filter with 30 dB attenuation in the stop band. The cutoff frequency f_{T,s} can be determined by the highest band pass filter upper edge frequency of the next filter bank plus an additional offset. The low pass filter prior to the pooling layer (decimation block) helps to avoid aliasing effects. The output of the pooling layer is the subsampled sub band envelope signal x_{R,i}(n), which then is processed through the non-linear function block. This non-linear function can include, for example, range limitation, normalization and further non-linear functions such as logarithms or exponentials. The output Y_{TF} of this stage is a N_{TF}×N_{R} matrix with

$${N}_{R} = ⌊ \frac{N}{R} ⌋$$

where R is a decimation factor and └.┘ is the floor operation.

The output signals y_{R,i}=[y_{R,i}(1) y_{R,i}(2) . . . y_{R,i}(N_{R})] are arranged into a matrix Y_{TF}=[y_{R,1}y_{R,2}^{T} . . . y_{R,N}_{_{TF}}^{T}]^{T} where each row corresponds to a specific frequency band. The output of this layer y_{TF} (where each row corresponds to a specific frequency band) is first fed row by row to N_{M} recurrent convolutional layers which can represent a bank of modulation filters. The modulation filters can be individually parameterized for each frequency band yielding an overall number of filters N_{M}×N_{TF}. The ordering of the parallel band pass filters for each frequency is analogous to the parallel band pass. The absolute values of the filtered signals \hat{x}_{M} (n) with i∈{1, . . . N_{TF}, ×N_{M}} of these filter banks y_{M,i}n)=|\hat{x}_{M,i}(n)| are averaged and the final result is a feature vector Y_{MF} with dimensions N_{TF}×N_{M}. This feature vector is the output of the pre-processing neural network and input to the classification neural network.

The classification neural network may be for example a fully connected neural network layer, a linear discriminant analysis (LDA) classifier, or a more complex classification layer. The outputs of this layer are the predefined class labels Cᵢ or/and probabilities Pᵢ for them.

As explained above, the multi-layer neural network arrangement is iteratively optimized. First an initial setting for the pre-processing neural network is chosen and the feature vectors Y_{MF} for the Training Set 1 are calculated. For this feature vector, the classification neural network can be trained by a standard method such as back propagation or LDA. Then for Training Set 2, the corresponding class labels or/and probabilities are calculated and used to calculate an error vector that is input to the training approach of the pre-processing neural network. This yields a new setting for the pre-processing neural network. With this new setting, the next iteration of the training procedure starts.

The training of the pre-processing neural network optimizes it in the sense of minimizing an error function, minimizing the mismatch between the estimated class labels and the ground truth class labels. Instead of explicitly training the weights of the pre-processing neural network via a back propagation procedure (which is the state-of-the art algorithm for training neural networks), meta-parameters are optimized, for example with generic algorithms or model-based optimization approaches. This significantly reduces the number of tunable weights and also reduces the amount of training data needed due to lower weight vector dimensionality. As a result the neural network has better generalization capabilities, which are important for its performance in previously unseen conditions.

The meta-parameters could be, for example, filter bandwidths and the neural network weights would be the coefficients of the corresponding filters. In this example, any filter design rule can be applied for computing the filter coefficients. However, other rules for mapping meta-parameters to network weights may be used as well. This mapping could be learned automatically via an optimization procedure and/or may be adaptive such that the network weights are updated during optimization and/or during the operation of the trained network. The optimal bandwidths of the filter for a given classification problem can be found by known optimization algorithms. Before running the optimization process, a filter design rule is chosen for mapping meta-parameters to filter coefficients. For example, Butterworth filters can be chosen for the first filter bank and Chebychev 2 filters for the second one, or vice versa.

FIG. 10 shows an example of how filter bank filter bandwidths may be structured according to an embodiment of the present invention. The first filters in the filter banks are low pass filters where the edge frequency is the lower edge frequency of the successive band pass filter and so on. This mapping rule from meta-parameters to network weights ensures that the network uses all information available in the input signal. The specification of the network structure via meta-parameters and filter design rules reduces the optimization complexity. The upper and lower edge frequencies of each filter can also be independently trained and other design rules are possible. With this approach, the initialization of the pre-processing neural network can be done by selection of all boundary frequencies according to

$$0 = {f}_{{u}_{0}} < {f}_{{u}_{1}} = {f}_{{l}_{2}} < ⋯ < {f}_{{l}_{N}} \leq \frac{f s}{2} ,$$

where fs is sampling frequency of corresponding input signal. The network weights can be achieved by using the defined mapping rule.

As mentioned above, there are N_{TF}·(N_{M}+1)−1 independently tunable parameters.

Finding optimal parameters using an exhaustive search may not be feasible due to the high dimensionality. A gradient descent algorithm also may not be suitable because the multimodal cost function (classification error) is not differentiable. Thus a Covariance Matrix Adaptation Evolution Strategy (CMA-ES) can be used based in order to find an ideal parameter set for the feature extraction step (see e.g., N. Hansen, “The CMA evolution strategy: A comparing review,” in Towards a new evolutionary computation. Advances in estimation of distribution algorithms. Springer, 2006, pp. 75-102, which is incorporated herein by reference in its entirety). ES is a subclass of evolutionary algorithms (EA) and shares the idea of imitating natural evolution, for instance by mutation and selection, and it does not require the computation of any derivatives (H. Beyer, Theory of Evolution Strategies, Springer, 2001 edition; incorporated herein by reference in its entirety). The optimal parameter set can be iteratively approximated by evaluating a fitness function after each step, where the fitness function or cost function may be the classification error (the ratio of the number of misclassified objects to the number of all objects) of the LDA classifier as a function of the independently tunable parameters.

The basic equation for CMA-ES is the sampling equation of new search points (Hansen 2006):

xₖ^{y+1}˜m^{(g)}+σ^{(g)} [symbol] (0, C^{(g)} for k=1, . . . ,λ

where g is the index of the current generation (iteration), xₖ^{g+1} is the i-th offspring from generation g+1, λ is the number of offspring, m^{(g)} is the mean value of the search distribution at generation g, [symbol] (0, C^{(g)}) is a multivariate normal distribution with the covariance matrix c^{(g)} of generation g, and σ^{(g)} is the step-size of generation g. From the λ sampled new solution candidates, the μ best points (in terms of minimal cost function) are selected and the new mean of generation g+1 is determined by a weighted average according to:

$${m}^{( g + 1 )} = {\sum }_{i = 1}^{μ} {ω}_{i} {x}_{i : μ}^{g + 1}$$

$${\sum }_{i = 1}^{μ} {ω}_{i} = 1 , {ω}_{1} \geq {ω}_{2} \geq ⋯ \geq {ω}_{μ} > 0$$

In each iteration of the CMA-ES, the covariance matrix C and the step-size a are adapted according to the success of the sampled offspring. The shape of the multivariate normal distribution is formed in the direction of the old mean m^{(g)} towards the new mean m^{(g+1)}. The sampling, selection and recombination steps are repeated until reaching either a predefined threshold on the cost function or a maximum number of generations, or the range of the current functional evaluation is below a threshold (local minima is reached). The allowed search space of the parameters can be restricted to intervals as described by Colutto et al. in S. Colutto, F. Frühauf, M. Fuchs, and O. Scherzer, “The CMA-ES on Riemannian manifolds to reconstruct shapes in 3-D voxel images,” IEEE Transactions on Evolutionary Computation, vol. 14, no. 2, pp. 227-245, April 2010, which is incorporated herein by reference in its entirety. For a more detailed description of CMA-ES, in particular on how the covariance matrix C and the step-size σ are adapted in each step, as well as a Matlab implementation, please refer to Hansen 2006. Other generic algorithms such as particle swarm optimization also can be used.

Optimizing the filter bank parameters used for deriving the weights of the network in order to decrease the classification error is a challenging task due to its high dimensionality and multi-modal error function. Brute-force and gradient-descent may not be feasible for this task. One useful approach may be based on Model-Based Optimization (MBO) (see Alexander Forrester, Andras Sobester, and Andy Keane. Engineering Design via Surrogate Modeling: A Practical Guide. Wiley, September 2008; and Claus Weihs, Swetlana Herbrandt, Nadja Bauer, Klaus Friedrichs, and Daniel Horn. Efficient Global Optimization: Motivation, Variations, and Applications. In ARCHIVES OF DATA SCIENCE, 2016, both of which are incorporated herein by reference in their entireties).

MBO is an iterative approach used to optimize a black box objective function. It is used where the evaluation of an objective function (e.g., the classification error depending on different filter bank parameters) is expensive in terms of available resources such as computational time. An approximation model, a so-called surrogate model, is constructed of this expensive objective function in order to find the optimal parameter for a given problem. The evaluation of the surrogate model is cheaper than the original objective function. The MBO steps can be divided as follows:
Design a sampling plan,
Constructing a surrogate model,
Exploring and exploiting a surrogate model.

A high dimensional multi-modal parameter space is assumed and the goal of the optimization is to find the point which minimizes the cost function. The initial step of the MBO is to construct a sampling plan. This means that n points are determined which will then be evaluated by the objective function. These n points should cover the whole region of the parameter space, and for this the space-filling design called Latin hypercube design can be used. The parameter space is divided into n equal-sized hyper-cubes (bins), where n∈{5 k, 6 k, . . . , 10 k} is recommended and k is the number of parameters. The points are then placed in the bins such that “from each occupied bin we could exit the parameter space along any direction parallel with any of the axes without encountering any other occupied bins” (Forrester 2008). Randomly set points do not guarantee the space-filling property of the sampling plan X (n×k matrix) and to evaluate the space-fillingness of X the maximin metric of Morris and Mitchell is used:
“We call X the maximin plan among all available plans if it maximizes d₁, among plans for which this is true, minimizes j₁, among all plans for which this is true, maximizes d₂, among all plans for which this is true, minimizes J₂, . . . , minimizes Jₘ.”
   With d₁, d₂, d₃, . . . , dₘ the list of unique values of distances between all possible pairs of points in the sampling plan X sorted in ascending order, and Jⱼ is the number of pairs of points in X separated by the distance dⱼ.

The above definition means that one sequentially maximizes d₁ and then minimizes J₁, maximizes d₂ and then minimizes J₂ and so on. Or in other words, the goal is to have as minimal distinct pairs with maximum distance as possible. As a metric for the distance d between two points the p-norm is used:

$${d}_{p} ( {x}^{( {i}_{1} )} , {x}^{( {i}_{2} )} ) = {( {\sum }_{j = 1}^{k} \vert {ϰ}_{j}^{{i}_{1}} - {ϰ}_{j}^{{i}_{2}} {\vert}^{p} )}^{1 / p}$$

where p=1 is used as the rectangular norm. Based on the above definition of a maximin plan, Morris and Mitchell propose comparing sampling plans according to the criterion:

$${Φ}_{q} ( X ) = {( {\sum }_{j = 1}^{m} {J}_{j} {d}_{j}^{- q} )}^{1 / q}$$

The smaller Φ_{q}, the better X fulfills the space-filling property (Forrester 2008). For the best Latin hypercube, Morris and Mitchell recommend minimizing Φ_{q} for q=1, 2, 5, 10, 20, 50 and 100 and choosing the sampling plan with the smallest Φ_{q}.

A surrogate model ĝ(x) can be constructed such that it is a reasonable approximation of the unknown objective function ƒ(x) (where x is a k dimensional vector pointing to a point in the parameter space). Different types of models can be constructed such as an ordinary Kriging model:

ĝ(x)=μ+Z(x)

where μ is a constant global mean and Z(x) is a Gaussian process. The mean of this Gaussian process is 0, and its covariance is:

Cov(Z(x),Z(x))=σ²ρ(x−x′,Ψ)

with ρ the Matern 3/2 kernel function and Ψ a scaling parameter. The constant σ² is global variance. The Matern 3/2 kernel is defined as:

$$ρ ( x - {x}^{′} ) = ( 1 + \sqrt{3} \frac{\vert x - {x}^{′} \vert}{Ψ} ) exp ( - \sqrt{3} \frac{\vert x - {x}^{′} \vert}{Ψ} )$$

So the unknown parameters of this model are μ, σ² and Ψ, which are estimated by using the n previously by the objective function evaluated points y=(y₁, . . . , yₙ)^{Γ}.

The likelihood function is:

$$L ( μ , {σ}^{2} , Ψ ) = \frac{1}{\sqrt{{( 2 π )}^{n} {σ}^{2 n} det ( R )}} exp ( - \frac{3}{2 {σ}^{2}} {( y - 1 μ )}^{T} {R}^{- 1} ( y - 1 μ ) )$$

with R(Ψ)=(ρ(xᵢ−xⱼ,Ψ))_{i,j=1, . . . , n} and det(R) its determinant. From this the Maximum likelihood estimation of the unknown parameters can be determined:

$$μ = {arg max}_{μ} L ( μ , {σ}^{2} , Ψ )$$

$${σ}^{2} = {arg max}_{{σ}^{2}} L ( μ , {σ}^{2} , Ψ )$$

$$Ψ = {arg max}_{Ψ} L ( μ , {σ}^{2} , Ψ )$$

The surrogate prediction \hat{f}ₙ(x) and the corresponding prediction uncertainty ŝₙ(x) (see Weihs 2016) can be determined based on the first n evaluations of f. The estimated surrogate function follows a normal distribution ĝ(x)˜ [symbol] (\hat{f}ₙ(x),ŝₙ²(x)). With the actual best value

$${y}_{min} = {min}_{i = 1 , \text{...} , n} {y}_{i} = {min}_{i = 1 , \text{...} , n} f ( {x}_{i} ) ,$$

then the improvement for a point x and the estimated surrogate ĝ(x) is Iₙ(x)=max(yₘᵢₙ−ĝ(x),0). The next point to evaluate is found by maximizing the expected improvement:

$${ϰ}_{n + 1} = {arg max}_{ϰ} E ( {I}_{n} ( ϰ ) )$$

The above criterion gives a balance between exploration (improving global accuracy of the surrogate model) and exploitation (improving local accuracy in the region of the optimum of the surrogate model). This ensures that the optimizer will not get stuck in local optima and yet converges to an optimum. After each iteration of MBO, the surrogate model will be updated. Different convergence criteria could be chosen to determine when to stop evaluating new points for updating the surrogate model. Some criteria could be, e.g., to define a preset number of iterations and stop after this or to stop after the expected improvement drops below a predefined threshold.

The hearing implant may be, without limitation, a cochlear implant, in which the electrodes of a multichannel electrode array are positioned such that they are, for example, spatially divided within the cochlea. The cochlear implant may be partially implanted, and include, without limitation, an external speech/signal processor, microphone and/or coil, with an implanted stimulator and/or electrode array. In other embodiments, the cochlear implant may be a totally implanted cochlear implant. In further embodiments, the multi-channel electrode may be associated with a brainstem implant, such as an auditory brainstem implant (ABI).

Embodiments of the invention may be implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

What is claimed is:

1. A signal processing method for generating stimulation signals for a hearing implant implanted in a patient, the method comprising:
classifying an audio input signal from an audio scene with a multi-layer neural network, the classifying comprising:
a) pre-processing the audio input signal with a pre-processing neural network using initial classification parameters to produce an initial signal classification, and
b) processing the initial scene classification with a scene classifier neural network using scene classification parameters to produce an audio scene classification output,
wherein the initial classification parameters reflect neural network training based on a first set of initial audio training data, and the scene classification parameters reflect neural network training on a second set of classification audio training data separate and different from the first set of initial audio training data;
processing the audio input signal and the audio scene classification output with a hearing implant signal processor for generating the stimulation signals.

2. The method according to claim 1, wherein the pre-processing neural network includes successive recurrent convolutional layers.

3. The method according to claim 2, wherein the recurrent convolutional layers are implemented as recursive filter banks.

4. The method according to claim 1, wherein the pre-processing neural network includes an envelope processing block configured for calculating sub-band signal envelopes for the audio input signal.

5. The method according to claim 1, wherein the pre-processing neural network includes a pooling layer configured for signal decimation within the pre-processing neural network.

6. The method according to claim 1, wherein the initial signal classification is a multi-dimensional feature vector.

7. The method according to claim 1, wherein the scene classifier neural network comprises a fully connected neural network layer.

8. The system according to claim 1, wherein the scene classifier neural network comprises a linear discriminant analysis (LDA) classifier.

9. A signal processing system for generating stimulation signals for a hearing implant implanted in a patient, the system comprising:
an audio scene classifier comprising a multi-layer neural network configured for classifying an audio input signal from an audio scene, wherein the audio scene classifier includes:
c) a pre-processing neural network configured for pre-processing the audio input signal based on initial classification parameters to produce an initial signal classification, and
d) a scene classifier neural network configured for processing the initial scene classification based on scene classification parameters to produce an audio scene classification output,
wherein the initial classification parameters reflect neural network training based on a first set of initial audio training data, and the scene classification parameters reflect neural network training on a second set of classification audio training data separate and different from the first set of initial audio training data;
a hearing implant signal processor configured for processing the audio input signal and the audio scene classification output for generating the stimulation signals.

10. The system according to claim 9, wherein the pre-processing neural network includes successive recurrent convolutional layers.

11. The system according to claim 10, wherein the recurrent convolutional layers are implemented as recursive filter banks.

12. The system according to claim 9, wherein the pre-processing neural network includes an envelope processing block configured for calculating sub-band signal envelopes for the audio input signal.

13. The system according to claim 9, wherein the pre-processing neural network includes a pooling layer configured for signal decimation within the pre-processing neural network.

14. The system according to claim 9, wherein the initial signal classification is a multi-dimensional feature vector.

15. The system according to claim 9, wherein the scene classifier neural network comprises a fully connected neural network layer.

16. The system according to claim 9, wherein the scene classifier neural network comprises a linear discriminant analysis (LDA) classifier.
